(19) United States
(12) Patent Application Publication
(10) Pub. No.: US 2026/0048250 A1
(43) Pub. Date: Feb. 19, 2026

(54) MICRONEEDLE DEVICE AND MICRONEEDLE SYSTEM USING THE SAME

(71) Applicant: Curiosis Inc., Seoul (KR)

(72) Inventors: Kyuntae KIM, Seoul (KR); Heewon LEE, Seoul (KR)

(73) Assignee: Curiosis Inc., Seoul (KR)

(21) Appl. No.: 19/087,893

(22) Filed: Mar. 24, 2025

(30) Foreign Application Priority Data
Mar. 26, 2024 (KR) 10-2024-0041354
Mar. 13, 2025 (KR) 10-2025-0033009

(51) Int. Cl. A61M 37/00

(52) U.S. Cl. CPC A61M 37/0015; A61M 2037/0023; A61M 2037/003; A61M 2037/0061; A61M 2205/07; A61M 2205/502; A61M 2210/04

(57) ABSTRACT

A microneedle device includes a housing; a needle assembly including a plurality of microneedles; a driver configured to generate a driving force for operating the needle assembly forward and backward; a controller configured to control an operation of the microneedle device; a needle cover configured to entirely or partially enclose the needle assembly; and a close-contact member provided in a front portion of the needle cover and configured to bring into contact with a skin of a user when using the microneedle device. The close-contact member may be formed of an elastically deformable material, and a front portion of the close-contact member is formed in a cup-like structure with a recess. Perforated portions may be provided in a central portion of the close-contact member such that the plurality of microneedles move into and out of the microneedle device via the perforated portions.

### CROSS-REFERENCE TO RELATED APPLICATIONS

This application claims priority to Korean Patent Application No. 10-2024-0041354 filed on Mar. 26, 2024 and Korean Patent Application No. 10-2025-0033009 filed on Mar. 13, 2025, the entire contents of which are herein incorporated by reference.

### TECHNICAL FIELD

The present disclosure relates to a microneedle device which forms fine holes in a skin with microneedles and a microneedle system using the same, and more particularly, to a microneedle device configured to improve an operational effect by providing a close-contact member at a front portion thereof, and a microneedle system using the same.

### BACKGROUND

A health condition of a skin has a large impact on the appearance. Thus, in recent years, various treatments have been applied onto the skin to lighten the skin, improve wrinkles, moisturize the skin, increase resilience of the skin, and the like, in addition to treat skin's diseases.

As is well known, the human skin may be roughly classified into an epidermal layer (epidermis), a dermal layer (dermis) and a fat layer. Among these, the dermal layer occupying a substantial portion of the skin is composed of a papillary layer and a plexiform layer. Capillaries, lymphatic tubes and the like are located in the papillary layer. Collagens as collagen fibers associated with wrinkles in the skin, elastin as elastic fibers that give elasticity to the skin, a substrate and the like are contained in the plexiform layer.

A visual condition of the skin is greatly dependent on a health status of the dermal layer. Accordingly, various treatments implemented for improving the condition of the skin are often targeted for the dermal layer.

Meanwhile, since the dermal layer is protected by the epidermal layer as described above, even if effective ingredients to be delivered to the dermal layer are applied onto the skin, an amount and injection speed of the effective ingredients that reach the dermal layer may not be sufficient.

### SUMMARY

The present disclosure was made to solve the above-mentioned matters, and the present disclosure is for the purpose of providing a microneedle device configured to be able to improve an operational effect by providing a close-contact member at a front portion thereof, and a microneedle system using the same.

Representative configurations of the present disclosure to achieve the above aspects are described below.

A microneedle device according to an example embodiment of the present disclosure may include; a housing; a needle assembly including a plurality of microneedles; a driver configured to generate a driving force for operating the needle assembly forward and backward; a controller configured to control an operation of the microneedle device; a needle cover configured to entirely or partially enclose the needle assembly; and a close-contact member provided in a front portion of the needle cover and configured to bring into contact with a skin of a user when using the microneedle device. According to an example embodiment of the present disclosure, the close-contact member may be formed of an elastically deformable material, and a front portion of the close-contact member is formed in a cup-like structure with a recess. According to an example embodiment of the present disclosure, perforated portions may be provided in a central portion of the close-contact member such that the plurality of microneedles move into and out of the microneedle device via the perforated portions.

In an aspect, the close-contact member may be configured to cover the front portion of the needle cover, and the perforated portions of the close-contact member may be formed as a plurality of through-holes provided in a central portion of the close-contact member configured to cover the front portion of the needle cover. Further, according to an example embodiment of the present disclosure, the close-contact member may include an axial extension portion that extends in an axial direction, and the plurality of through-holes may be provided in the axial extension portion.

In an aspect, the axial extension portion may be formed to extend backward from the close-contact member in the axial direction.

In an aspect, the close-contact member may be configured to be deformed when the microneedle device is pressed toward the skin of the user such that a bottom surface of the recess is brought into close contact with the skin of the user.

In an aspect, each of the plurality of microneedles may be formed in a tubular structure having a perforated central portion.

In an aspect, the plurality of through-holes may be provided to correspond in number to the plurality of microneedles provided in the needle assembly.

In an aspect, each of the plurality of through-holes may have a diameter of 0.25 millimeter (mm) to 3.0 mm.

In an aspect, the microneedle device may further include: a pump unit configured to generate compressed air and supply the compressed air to an interior of the needle assembly; and a valve configured to open and close a pneumatic hose provided between the pump unit and the needle assembly.

In an aspect, an opening/closing operation of the valve may be controlled by the controller. When the valve is brought into an open state, the compressed air may be supplied from the pump unit to the interior of the needle assembly such that a positive pressure is formed in an inner space portion of the needle cover.

In an aspect, the controller may be configured to control the opening/closing operation of the valve based on information input from the user.

In an aspect, the controller may be configured to control power of the pump unit based on the information input from the user.

In an aspect, before the positive pressure is formed in the inner space portion of the needle cover, a negative pressure may be formed in the inner space portion of the needle cover.

In an aspect, the controller may be configured to control at least one needling operation in which the plurality of microneedles is penetrated into the skin and subsequently return to original positions thereof. The controller may be configured to control the microneedle device in a stacking mode in which a penetration depth of each of the plurality of microneedles is set for each of the at least one needling operation.

In an aspect, the stacking mode may be set such that the at least one needling operation is repeated twice or more times at a same skin position or at different time intervals. In the stacking mode, the at least one needling operation may be performed at a same penetration depth or different penetration depths.

According to another example embodiment of the present disclosure, a microneedle system may include: the aforementioned microneedle device; and a display device in communication with the controller of the microneedle device and configured to provide a user interface for receiving setting information about the operation of the microneedle device from a user and configured to transmit the setting information input from the user to the controller of the microneedle device.

Further, the microneedle device according to the present disclosure and the microneedle system using the same may further include other additional configurations without departing from the technical sprit of the present disclosure.

A microneedle device according to an example embodiment of the present disclosure is configured to form fine holes in a skin with microneedles and subsequently inject effective ingredients into the skin via the fine holes. This makes it possible to inject a sufficient amount of the effective ingredients into the dermal layer, thereby improving skin regeneration and treatment effects while minimizing damage to the skin.

Further, a microneedle device according to an example embodiment of the present disclosure is configured such that effective ingredients applied onto the skin may be introduced into and held in a perforated portion provided in a central portion of a close-contact member. This makes it possible to further increase the amount of the effective ingredients injected into the skin.

Further, a microneedle device according to an example embodiment of the present disclosure is configured to inject effective ingredients applied onto the skin into the skin using compressed air while alternately generating a negative pressure and a positive pressure with a pump unit and a valve. This makes it possible to more easily penetrate the effective ingredients applied onto the skin into the dermal layer in a more stable manner.

Further, a microneedle device according to an example embodiment of the present disclosure is configured such that microneedles repeatedly penetrates into the skin multiple times in a stacking mode. This makes it possible to treat several layers in the skin (for example, a papillary layer, a plexiform layer, and the like) at once by a mechanism of micro-needling and the injection of effective ingredients.

### BRIEF DESCRIPTION OF THE DRAWINGS

FIG. 1 schematically illustrates an overall configuration of a microneedle system according to an example embodiment of the present disclosure.

FIG. 2 exemplarily illustrates a basic structure of a microneedle device according to an example embodiment of the present disclosure.

FIG. 3 exemplarily illustrates an internal structure of the microneedle device illustrated in FIG. 2.

FIG. 4 exemplarily illustrates a cross-sectional structure of a front portion of the microneedle device illustrated in FIG. 2.

FIG. 5 schematically illustrates a principle of injecting effective ingredients into a skin by microneedles of a hollow structure in the microneedle device according to an example embodiment of the present disclosure.

FIG. 6 exemplarily shows a structure of the front portion of the microneedle device (an example embodiment of the microneedle device including a close-contact member provided in the front portion) according to an example embodiment of the present disclosure.

FIGS. 7A to 7D exemplarily illustrate an operation principle of the microneedle device illustrated in FIG. 6.

FIG. 8 exemplarily illustrates how a pump unit and a valve operate to generate a negative pressure and a positive pressure in the microneedle device according to an example embodiment of the present disclosure.

FIGS. 9A and 9B schematically illustrate a state in which the microneedle is inserted into the skin in the microneedle device according to an example embodiment of the present disclosure (FIG. 9A illustrates a state in which a final penetration depth is set to 4 millimeter (mm) and the microneedle is penetrated by a depth of 3 mm, and FIG. 9B illustrates a state in which the microneedle is penetrated by 4 mm, which is the final penetration depth, and subsequently, is retracted by 1 mm).

FIG. 10 exemplarily illustrates a method of injecting effective ingredients into the skin with the microneedle device according to an example embodiment of the present disclosure.

FIGS. 11 and 12 exemplarily illustrate a user interface for receiving setting information input from a user to implement a stacking mode operation in the microneedle system according to an example embodiment of the present disclosure (FIG. 11 illustrates an interface for receiving time interval information input from the user, and FIG. 12 illustrates an interface for receiving information about a penetration depth for each needling operation from the user).

### DETAILED DESCRIPTION

Example embodiments of the present disclosure are exemplified for the purpose of describing the technical spirit of the present disclosure. The scope of the claims according to the present disclosure is not limited to the example embodiments described below or to the detailed descriptions of these example embodiments.

Unless otherwise defined, all technical and scientific terms used herein have the same meaning commonly understood by those skilled in the art to which the present disclosure pertains. All terms used herein are selected for the purpose of more clearly describing the present disclosure and not limiting the scope of the present disclosure defined by appended claims.

Unless the phrase or sentence clearly indicates otherwise, terms “comprising” “including” “having” and the like used herein should be construed as open-ended terms encompassing the possibility of including other example embodiments.

The terms “front,” “tip end,” and the like used herein mean a direction in which a skin is located relative to a microneedle device, and the terms “back,” “rear end,” and the like used herein mean a direction opposite the direction.

Further, the term “skin” used herein should be understood as the entire skin including a scalp, and a microneedle device of the present disclosure may also be used to inject effective ingredients into the scalp.

The term “effective ingredients” used herein may be understood in a sense including all substances aimed at a physiological function regulation and a biological process. All substances may be interpreted to include drugs (pharmaceuticals) that exhibit pharmacological effects, as well as any ingredients for cosmetic purposes (skin improvement, anti-aging, skin regeneration, and the like) (for example, cosmetic, platelet-rich plasma (PRP), and the like).

The singular form described herein may include the plural form unless the context clearly dictates otherwise, and this is equally applied to the singular form set forth in the claims.

Throughout the present specification, when a constituent element is referred to as being “positioned” at or “formed” on one side of another constituent element, the constituent element may be in direct contact with or directly formed on the one side of another constituent element, or may be positioned at or formed on another constituent element by intervening yet another constituent element therebetween.

In addition, the terms such as “part”, “module”, “unit” and the like used herein may refer to a unit that performs at least one function or operation, which may be realized as hardware or software, or may be realized as a combination of hardware and software.

The expression “configured (or set) to - - - ” described herein may be alternatively expressed to, for example, “suitable for - - - ,” “having the capacity to - - - ,” “designed to - - - ,” “adapted to - - - ,” “made to - - - ,” “capable of - - - ,” or the like depending on situations. The expression “configured (or set) to - - - ” may not necessarily mean only “specifically designed to - - - ” in hardware. Alternatively, in some circumstances, the expression “system configured to - - - ” may mean that the system is “capable of - - - ” together with other apparatus(es) or constituent element(s). The phrase “processor configured (or set) to perform A, Band C” may mean, for example, a dedicated processor (for example, embedded process) configured to perform a respective operation, or a generic-purpose processor (for example, a central processing unit (CPU) or an application processor) capable of performing respective operations by executing one or more software programs stored in a memory.

Further, although the terms including ordinal numbers such as a first, a second and the like used herein may be used to describe various constituent elements, the order or importance of the respective constituent elements is not limited by these terms unless otherwise defined.

Hereinafter, example embodiments of the present disclosure will be described in detail with reference to the accompanying drawings at such an extent that they may be readily practiced by those ordinary skilled in the art. In the accompanying drawings, the same reference numerals are assigned to the same or corresponding components. Further, in the following descriptions of the example embodiments, duplicate descriptions of the same or corresponding constituent elements may be omitted. However, even though a description of a constituent element is omitted, such a constituent element is not intended to be excluded in any example embodiment.

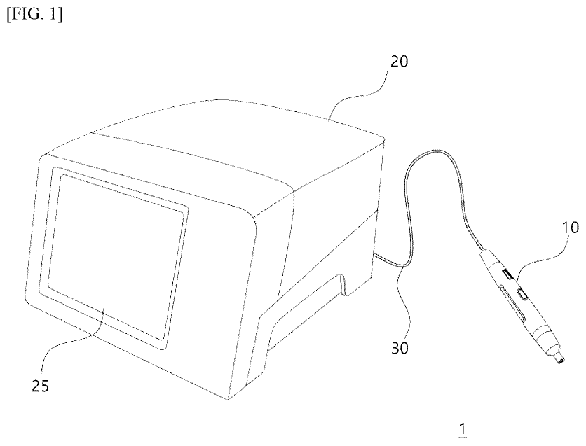

Referring to FIG. 1, a schematic view of a microneedle system 1 according to an example embodiment of the present disclosure is exemplarily illustrated.

As illustrated in FIG. 1, the microneedle system 1 may be configured to include a microneedle device (handpiece) 10, a display device 20, a cable 30 that connects the microneedle device 10 and the display device 20, and the like. The microneedle system 1 according to an example embodiment of the present disclosure may be used for the purpose of lightening the skin, improving winkles, moisturizing the skin, increasing resilience of the skin, treating scars (for example, acne scars), or the like.

The microneedle device 10 is a major constituent element of the present disclosure, which will be described in detail below.

The display device 20 may include a user interface 25 (for example, a touch screen) to receive setting information about an operation of the microneedle device 10 from a user, and may transfer the setting information input by the user to a controller 400 of the microneedle device 10 via the cable 30.

Examples of the setting information transferred to the microneedle device 10 may include a final penetration depth of a microneedle, a positive-pressure generation time point (for example, a penetration depth of the microneedle when the positive pressure is generated), power of a pump unit, an operation mode of the microneedle device 10, a time interval for an automatic operation mode, a penetration depth during a needling operation, and the like.

Hereinafter, a configuration of the microneedle device 10 according to an example embodiment of the present disclosure will be described and subsequently, a method of operating the microneedle device 10 according to an example embodiment of the present disclosure will be described.

### 1. Configuration of Microneedle Device According to Example Embodiment of Present Disclosure

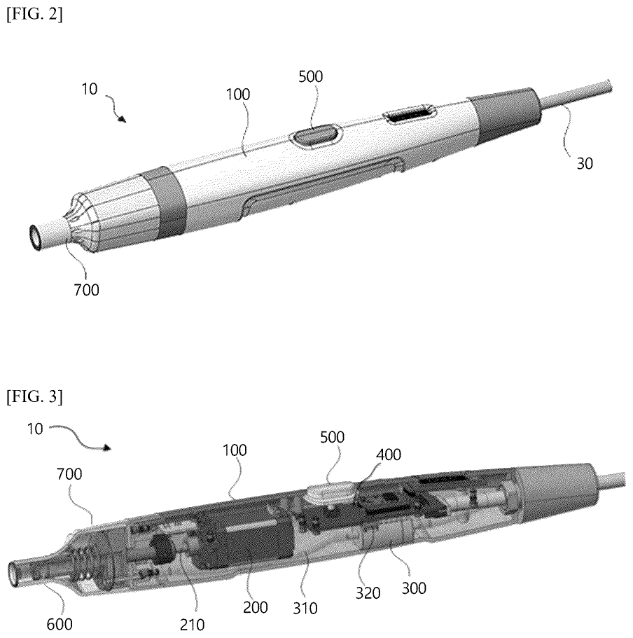

Referring to FIGS. 2 and 3, there are exemplarily illustrated a basic structure of the microneedle device 10 according to an example embodiment of the present disclosure.

As illustrated in FIGS. 2 and 3, the microneedle device 10 according to an example embodiment of the present disclosure may include a housing 100, a driver 200, a pump unit 300, the controller 400 and the like, which are accommodated in the housing 100, and may be configured to operate a needle assembly 600 mounted in front of the microneedle device 10 forward and backward.

According to an example embodiment of the present disclosure, the housing 100 is a part which forms an outer shape of the microneedle device 10, and may be formed of a single member, or may have a structure in which a plurality of members is coupled to each other.

According to an example embodiment of the present disclosure, the needle assembly (microneedle unit) 600 described below may be configured to be coupled to a tip end of the housing 100. The cable 30 described above may be configured to be connected to a rear end of the housing 100.

According to an example embodiment of the present disclosure, the driver 200 is a part which generates and supplies a driving force for operating the microneedle device 10. A linear motor or the like may be used as the driver 200 of the microneedle device 10 according to an example embodiment of the present disclosure.

Unlike a typical electric motor configured to generate a rotational force as a rotor rotates inside a stator, the linear motor is configured so that a mover moves linearly relative to a deployed stator. This makes it possible to easily implement a linear reciprocating motion in a narrow space.

According to an example embodiment of the present disclosure, the pump unit 300 is configured to compress air at a predetermined pressure and supply the same. The pump unit 300 has a function of supplying the compressed air to the needle assembly 600 described below so that effective ingredients may be easily penetrated into a skin by the compressed air.

According to an example embodiment of the present disclosure, a pneumatic hose 310 may be connected between the pump unit 300 and the needle assembly 600. The compressed air generated in the pump unit 300 may be transferred to the needle assembly 600 via the pneumatic hose 310. A valve 320 may be provided in the pump unit 300. By opening and closing the valve 320, a certain amount of compressed air may be supplied to the needle assembly 600 only when needed.

According to an example embodiment of the present disclosure, the valve 320 provided in the pump unit 300 may be electrically connected to the controller 400. An operation of the valve 320 may be controlled by the controller 400. An example of the valve 320 may include a solenoid valve or the like.

According to an example embodiment of the present disclosure, the controller 400 controls the operation of the microneedle device 10 according to an example embodiment of the present disclosure and may be formed in the form of a printed circuit board (PCB) assembly using a printed circuit board.

According to an example embodiment of the present disclosure, an operation unit 500 may be provided on one side of the housing 100. A user may operate the microneedle device 10 with the operation unit 500.

According to an example embodiment of the present disclosure, the operation unit 500 may be formed in the form of an operation button that the user may press with his/her finger(s). With this configuration, the user may easily operate the operation unit 500 with his/her finger(s) while holding the microneedle device 10 by his/her hand.

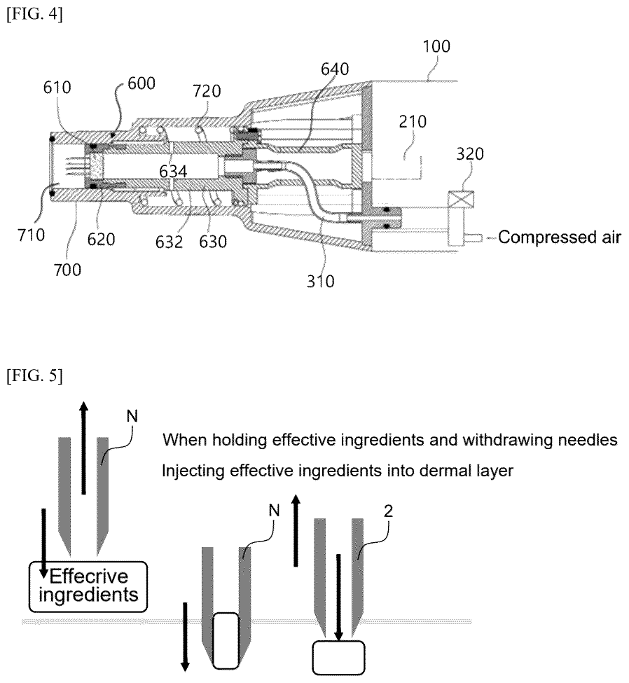

Referring to FIG. 4, there are exemplarily illustrated a structure of a front portion of the microneedle device 10 according to an example embodiment of the present disclosure.

As illustrated in the figure, the needle assembly 600 may include a plurality of microneedles N which is inserted into a skin of a patient, and may have a function of forming fine holes in the skin of the patient by the microneedles N and efficiently injecting effective ingredients into the skin.

According to an example embodiment of the present disclosure, the plurality of microneedles N may be provided at predetermined intervals in a needle mounting plate 610. The plurality of microneedles N may be mounted and fixed to the needle mounting plate 610 while penetrating the needle mounting plate 610.

According to an example embodiment of the present disclosure, each of the microneedles N may be formed in an approximately tubular structure having a perforated central portion. An inclined bevel portion may be formed at a tip end of each microneedle N so that each microneedle N has a fine-tipped structure in which a tip of a front end portion is sharpened.

According to an example embodiment of the present disclosure, the needle assembly 600 may be formed to have a substantially cylindrical structure and may be coupled to a front portion of the driver 200 via a plunger 630, a connection tube 640, and the like, which will be described later.

According to an example embodiment of the present disclosure, the plunger 630 of the needle assembly 600 may be formed in a structure having a perforated central portion. The needle mounting plate 610 may be mounted on a front end portion of the plunger 630 and may be configured to cover one end of the open central portion of the plunger 630.

According to an example embodiment of the present disclosure, the needle mounting plate 610 of the needle assembly 600 may be fixed to the front end portion of the plunger 630.

For example, in a case of an example embodiment illustrated in the figures, the needle mounting plate 610 of the needle assembly 600 may be mounted and fixed to the front end portion of the plunger 630 via a fixture 620.

Specifically, in the example embodiment illustrated in the figures, the fixture 620 has a stepped seat portion formed at a rear side thereof. The needle mounting plate 610 is coupled and fixed to the front end portion of the plunger 630 in a state in which the needle mounting plate 610 is coupled to the stepped seat portion.

With this configuration, the front end portion of the plunger 630 may be closed by the needle mounting plate 610 and air may flow over both sides of the needle mounting plate 610 via the microneedles N.

According to an example embodiment of the present disclosure, the plunger 630 may be a cylindrical member having an inner hollow portion 632. A rear end portion of the plunger 630 may be connected to the connection tube 640. A rear end of the connection tube 640 may be connected to a movable rod 210 of the driver 200.

With this configuration, when the movable rod 210 reciprocates forward and backward by the driver 200, the connection tube 640 and the plunger 630 connected to the movable rod 210 also moves forward and backward. As a result, the needle mounting plate 610 in which the microneedles N are mounted may reciprocate forward and backward.

According to an example embodiment of the present disclosure, the pneumatic hose 310 connected to the pump unit 300 may be mounted to penetrate the connection tube 640. A front end portion of the pneumatic hose 310 may be connected and fixed to the inner hollow portion 632 of the plunger 630 so that the pneumatic hose 310 is in communication with the plunger 630.

According to an example embodiment of the present disclosure, the pneumatic hose 310 mounted to penetrate the connection tube 640 may be formed to be flexible. Thus, when the connection tube 640 moves, the pneumatic hose 310 may also flexibly move to follow the movement of the connection tube 640, thereby smoothly guiding the movement of the connection tube 640.

According to an example embodiment of the present disclosure, an exhaust hole 634 may be formed in a periphery of the plunger 630. The inner hollow portion 632 of the plunger 630 may be in communication with the outside via the exhaust hole 634.

According to an example embodiment of the present disclosure, a needle cover 700 may be provided in the front end portion of the housing 100. The needle cover 700 may be configured to entirely or partially surround the needle assembly 600 (for example, the front end portion of the housing 100).

According to an example embodiment of the present disclosure, the needle cover 700 may be configured to have a space portion 710 at a central portion thereof. A front portion of the needle cover 700 may be formed in a forwardly-open structure.

According to an example embodiment of the present disclosure, the plunger 630 (and the needle mounting plate 610 and the connection tube 640 connected to the plunger 630) may be connected to the needle cover 700 via a spring 720.

For example, in the microneedle device 10 illustrated in the figures, one end of the spring 720 comes into contact with the needle cover 700 to be supported by the needle cover 700, and the other end of the spring 760 comes into contact with one side of the plunger 630 to be supported by the plunger 630. With this configuration, the plunger 630 (and the needle mounting plate 610 and the connection tube 640 connected to the plunger 630) may be elastically assembled with the needle cover 700 and the housing 100.

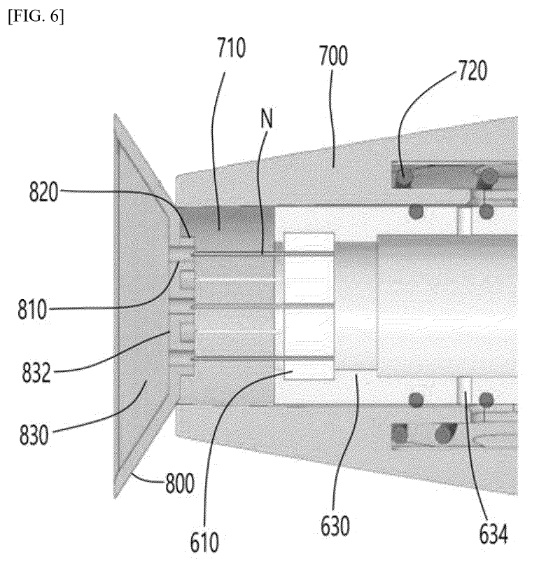

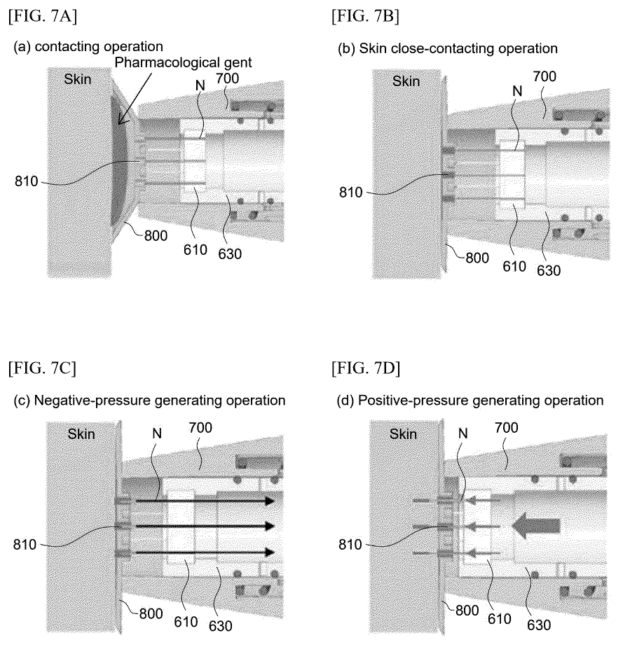

Further, according to an example embodiment of the present disclosure, as illustrated in FIGS. 6 and 7A to 7D, the front portion of the needle cover 700 may include a close-contact member 800 (configurations other than the close-contact member 800 may be implemented in substantially the same or similar manner as those described with reference to FIGS. 2 to 4).

According to an example embodiment of the present disclosure, the close-contact member 800 may be formed of an elastically deformable material, such as a rubber, and may be configured to bring into contact with the skin of the user when using the microneedle device 10.

According to an example embodiment of the present disclosure, a central portion of the close-contact member 800 may be perforated. The microneedles N may move into and out of the microneedle device 10 via such a perforated portion.

For example, as illustrated in FIGS. 6 and 7A to 7D, the close-contact member 800 may have a plurality of through-holes 810. The microneedles N may move into and out of the microneedle device 10 via the through-holes 810, respectively.

On the other hand, instead of the above-described structure in which the plurality of through-holes 810 are partially formed in the central portion, the close-contact member 800 may be formed in a structure in which the central portion is entirely perforated such that the space portion 710 of the needle cover 700 is open forward via the perforated central portion of the close-contact member 800.

With this configuration, when the microneedle device 10 is brought into close contact with the skin in a state in which the effective ingredients are applied onto the skin, the effective ingredients applied onto the skin may be introduced into and be held by the perforated portions (for example, the through-holes 810) provided in the close-contact member 800 (see FIGS. 7A to 7D). This makes it possible to inject a relatively large amount of the effective ingredients into the skin when penetrating the microneedles N into the skin.

In particular, a relatively large amount of the effective ingredients applied onto the skin may be introduced into and be held by the perforated portions (for example, the through-holes 810) provided in the close-contact member 800 due to a capillary phenomenon and hydroplaning phenomenon. The amount of the effective ingredients injected into the skin may be significantly increased.

According to an example embodiment of the present disclosure, the close-contact member 800 may include an axial extension portion 820 that extends in an axial direction (for example, backward in the axial direction). The above-described through-holes 810 may be defined in the axial extension portion 820.

As described above, when the through-holes 810 are formed in the axial extension portion 820, the amount of the effective ingredients that are introduced into and held by the through-holes 810 may be increased due to the capillary phenomenon and the hydroplaning phenomenon. This makes it possible to more effectively increase the amount of the effective ingredients injected into the skin.

According to an example embodiment of the present disclosure, the front portion of the close-contact member 800 may be formed in a cup-like structure in which a recess 830 is formed to be depressed backward along the axial direction.

As described above, when the recess 830 having such a depressed structure is formed in the front portion of the close-contact member 800, the effective ingredients applied onto the skin are received in and held by the recess 830. Accordingly, a relatively large amount of the effective ingredients may be held by the microneedle device 10 (see FIG. 7A). This makes it possible to more effectively increase the amount of the effective ingredients injected into the skin.

According to an example embodiment of the present disclosure, the close-contact member 800 may be configured to be elastically deformed when the microneedle device 10 is brought into close contact with the skin, so that a bottom surface 832 of the recess 830 is brought into close contact with the skin (see FIG. 7B).

In this case, the effective ingredients present in the recess 830 may be pushed into and held by the through-holes 810. When the microneedles N are penetrated into the skin, the effective ingredients may be injected into the skin.

According to an example embodiment of the present disclosure, the through-holes 810 may be provided to correspond to the number (for example, 1 to 50) of microneedles N provided in the needle assembly 600. Each of the through-holes 810 may have a diameter of about 0.25 mm to 3 mm.

### 2. Operating Principle of Microneedle Device According to Example Embodiment of Present Disclosure

A basic operating principle of the microneedle device 10 configured as above according to an example embodiment of the present disclosure is as follows.

Prior to specifically describe the operating principle, the microneedle device 10 according to an example embodiment of the present disclosure will be conceptually described with a focus on obvious differences from a technology in the related art.

As illustrated in FIG. 5, the microneedle device 10 according to an example embodiment of the present disclosure uses a method of injecting the effective ingredients into a dermal layer of the skin with the microneedles N instead of using an injection liquid in the state in which the effective ingredients are applied onto the skin.

As described above, the microneedle device 10 according to an example embodiment of the present disclosure does not use the injection liquid. Therefore, the present disclosure is not regulated by medical treatment laws. Thus, the present disclosure may be applied to various skin boosters other than limited pharmacological agents.

As illustrated in FIG. 5, the effective ingredients may be injected into the skin by applying a skin booster as the effective ingredients on the skin, followed by bring each microneedle N close to the skin so that the effective ingredients are received in the central hollow of each microneedle N, followed by penetrating each microneedle N into the skin, followed by blowing compressed air into the central hollow of each microneedle N at the time of penetrating each microneedle N into the skin or after each microneedle N penetrates into the skin, followed by removing each microneedle N from the skin.

Next, the needling operation of the microneedle device 10 according to an example embodiment of the present disclosure will be described in detail (as illustrated in FIG. 6, the needling operation will be described with reference to the example embodiment in which the close-contact member 800 is provided in the front portion of the microneedle device 10).

As illustrated in FIG. 7, a method of operating the microneedle device 10 according to an example embodiment of the present disclosure may include a skin contacting operation, a skin close-contacting operation, a negative-pressure generating operation and a positive-pressure generating operation.

First, the skin contacting operation is a preliminary operation of bringing the microneedle device 10 into contact with the skin in the state in which the effective ingredients are applied onto the skin. As illustrated in FIG. 7, in the skin contacting operation, the close-contact member 800 provided in the front portion of the microneedle device 10 is brought into contact with the skin.

At this time, the microneedles N are in a home position state. The expression “home position state” means a state in which ends of the microneedles N are positioned at an interval with respect to the tip end of the needle cover 700. In the home position state, the pump unit 300 is operated to generate the compressed air, but the supply of the compressed air is blocked by the valve 320.

As illustrated in FIG. 6, in the case in which the microneedle device 10 is configured such that the close-contact member 800 is provided in the front portion of the needle cover 700, the effective ingredients applied onto the skin are introduced into and received in the recess 830 provided in the front portion of the close-contact member 800 in the skin close-contacting operation (see FIG. 7A).

Subsequently, the skin close-contacting operation is an operation of pressing the microneedle device 10 to be in close contact with the skin. As described above, in the case in which the close-contact member 800 is provided in the front portion of the needle cover 700, the close-contact member 800 may be elastically deformed in the skin close-contacting operation so that the bottom surface 832 of the recess 830 is brought into close contact with the skin (see FIG. 7B).

At this time, the effective ingredients received in the recess 830 of the close-contact member 800 may be pushed into and held by the perforated portions (for example, the through-holes 810) provided in the close-contact member 800 (see FIG. 7B). The effective ingredients may be injected into the skin when the microneedles N are inserted into the skin.

As illustrated in FIG. 7B, the microneedles N are positioned at the home position even in the skin close-contacting operation.

The negative-pressure generating operation is an operation of forming a negative pressure in the space portion 710 of the needle cover 700 while moving the needle assembly 600 forward by the driver 200. When the negative pressure is generated in the space portion 710 of the needle cover 700, the effective ingredients applied onto the skin may be easily introduced into the hollow interior of each of the microneedles N (see FIG. 7C).

In particular, as illustrated in FIG. 6, in the configuration in which the close-contact member 800 is provided in the front portion of the needle cover 700, the effective ingredients that has been inserted into and held in the perforated portions (for example, the through-holes 810) of the close-contact member 800 may be introduced into the hollow interior of each of the microneedles Nin the negative-pressure generating operation. Accordingly, the microneedles N may penetrate into the skin while holding a relatively large amount of the effective ingredients.

At this time, the compressed air generated by the pump unit 300 remains blocked by the valve 320.

The positive-pressure generating operation is an operation of further moving the needle assembly 600 forward such that the microneedles N penetrate the skin while holding the effective ingredients, and opening the valve 320 to supply the compressed air generated from the pump unit 300 so that the effective ingredients are injected into the skin (see FIG. 7D).

According to an example embodiment of the present disclosure, the needle assembly 600 may be moved backward and the microneedles N may be retracted from the skin to further supply the compressed air for several seconds, so that a closed space is kept at a positive pressure.

A sequence of operations as described above may provide the same effects as those of directly injecting effective ingredients into the skin with needles. This makes it possible to inject the effective ingredients into the skin in a more smooth and efficient manner.

In this case, the spring 720 may perform a function of applying an elastic force so that the needle assembly 600 may smoothly return to the home position.

### 3. Control of Positive-Pressure Generation by Microneedle Device According to Example Embodiment of Present Disclosure

As described above, the microneedle device 10 according to an example embodiment of the present disclosure is configured to generate the positive pressure using the pump unit 300 and the valve 320, rather than generating the positive pressure through a change in volume by the movement of the plunger 630. Thus, it is possible to control a time point at which the positive pressure is generated independent of the movement of the plunger 630.

That is, the microneedle device 10 according to an example embodiment of the present disclosure may control the time point of the generation of the positive pressure independent of the movement of the plunger 630 by controlling the operation of the pump unit 300 and time points at which the valve 320 are open and closed.

Accordingly, depending on a condition of the skin to be treated, it is possible to optimize a profile in which the effective ingredients are injected into the skin (a profile in which the effective ingredients are injected to correspond to different depths of the skin). For example, the effective ingredients may be injected uniformly regardless of the different depths of the skin. Alternatively, the effective ingredients may be injected intensively at a specific depth. Such an action may be further maximized by adjusting power of the pump unit 300.

That is, by generating and maintaining a predetermined positive pressure as soon as the microneedles N begin to penetrate the skin (or before the penetration), it is possible to uniformly inject the effective ingredients regardless of the different depths of the skin. Alternatively, when the microneedles N reaches the specific depth, the compressed air of a relatively high pressure may be blown into the microneedles N to intensively inject the effective ingredients into the specific depth. In the case, the specific depth may be different from that at which the microneedles N will finally penetrate the skin (hereinafter, referred to as “final penetration depth”).

As described above with reference to FIG. 1, the user may input, as the setting information, a time point at which a desired positive-pressure is generated (the penetration depth of the microneedle at the time of generating the positive pressure, that is, the specific depth) and a desired power of the pump unit 300, with the user interface 25 of the display device 20. The setting information is transmitted to the controller 400 of the microneedle device 10. The controller 400 controls the opening/closing of the valve 320, the power of the pump unit 300 and the like based on the setting information.

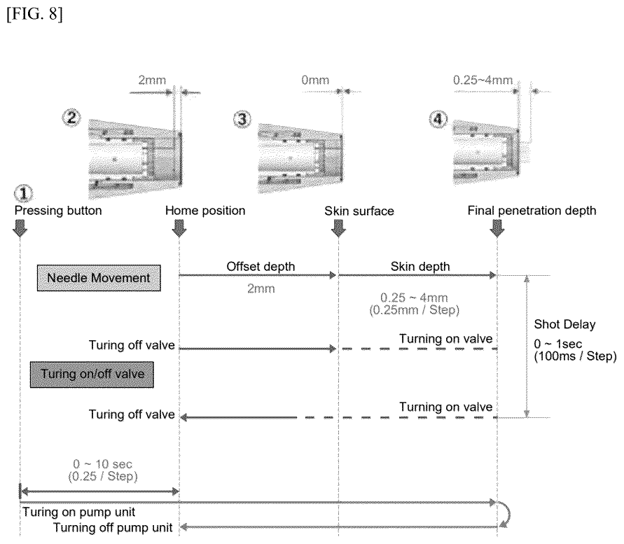

FIG. 8 illustrates a procedure of controlling the operation of the pump unit 300 and the opening/closing of the valve 320 according to an example embodiment of the present disclosure.

When the operation button of the operation unit 500 is pressed by the user, the microneedle device 10 starts to operate.

In an initial state of the microneedle device 10 before the user presses the operation button of the operation unit 500, the pump unit 300 remains turned off and the valve 320 also remains turned off (the valve 320 remains closed).

When the user presses the operation button of the operation unit 500, the pump unit 300 is turned on to generate the compressed air. The flow of the compressed air is blocked by the valve 320. At this time, the microneedles N are in the home position state (for example, the microneedles N are offset inward from the tip end of the needle cover 700).

When the operation button of the operation unit 500 is pressed by the user and a predetermined time period (about 10 seconds or less) elapses, the microneedles N starts to move forward. In the linear motor, since a movement distance of the movable rod 210 per pulse is set in advance, the movement distance of the movable rod 210, that is, a movement distance of the microneedles N may be determined based on the number of pulses.

When the microneedles N are moved by an offset distance, the microneedles N come into contact with the skin, and subsequently penetrate the skin. In the case, the offset distance may be set in a range of more than zero to 7 mm or less. The offset distance may be set according to a configuration of the needle assembly (microneedle unit) and user's requirement. For example, when the offset distance is set in increments of 0.25 mm in the range of more than zero to 7 mm or less, the offset distance may be set to 0.25 mm, 0.5 mm, 0.75 mm, and 1 mm. Even in the remaining range of more than 1 mm to 7 mm or less, the offset distance may be set in the same manner as the above.

In the microneedle device 10 according to an example embodiment of the present disclosure, the positive pressure is generated by turning (opening) the valve 320 on. That is, a time point at which the valve 320 is turned on may be the positive-pressure generation time point.

The profile in which the effective ingredients are injected to correspond to different depths of the skin may vary depending on the positive-pressure generation time point, a magnitude of the positive pressure, a type of effective ingredients (that is, skin booster) or the like. Further, a skin state at a site to be treated (for example, a position or state of the epidermal layer, a position or state of the dermal layer, or the like) varies depending on a person to be treated and a skin site of the person (forehead site, eye site or the like.).

In the microneedle device 10 according to an example embodiment of the present disclosure, the user may set the positive-pressure generation time point or the magnitude of the positive pressure in consideration of the type of skin booster, a skin condition of a site to be treated, and the like to obtain a profile in which desired effective ingredients are injected.

For example, when wanting effective ingredients to be intensively injected at a specific depth of 3 mm in consideration of a position of the epidermal layer or a position of the dermal layer at the skin site to be treated, the user may set the positive-pressure generation time point or the magnitude of the positive pressure corresponding thereto. The specific depth of 3 mm mentioned as above is merely an example. The specific depth may be set in a range of more than zero to 7 mm or less. For example, when the specific depth is set in increments of 0.25 mm in the range of more than zero to 1 mm or less, the specific depth may be set to 0.25 mm, 0.5 mm, 0.75 mm, and 1 mm. Even in the remaining range of more than 1 mm to 7 mm or less, the specific depth may be set in the same manner as the above.

According to an example embodiment of the present disclosure, the positive-pressure generation time point may be set to correspond to a penetration depth of the microneedles N at the positive-pressure generation time point so that the user may intuitively set the positive-pressure generation time point. For example, in a case in which the user sets to a depth of 3 mm as the positive-pressure generation time point (the penetration depth of the microneedles Nat the positive-pressure generation time point), when the penetration depth of the microneedles N is 3 mm, the valve 320 is open to generate the positive pressure.

Further, according to the profile in which the desired effective ingredients are injected, the positive-pressure generation time point may be set while the microneedles N move forward, or may be set while the microneedles N move backward.

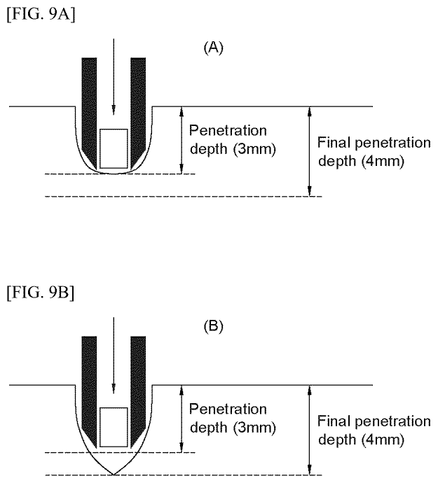

For example, FIG. 9A illustrates a state in which, when the final penetration depth is 4 mm, the microneedle N moves forward by 3 mm. FIG. 9B illustrates a state in which, when the final penetration depth is 4 mm, the microneedle N penetrates by 4 mm, which is the final penetration depth, and subsequently move backward by 1 mm. FIGS. 9A and 9B illustrate states in which, when the penetration depth of the microneedle N is 3 mm, shapes formed in the skin by the penetration of the microneedle N are different from each other. In this case, the profile in which the effective ingredients are injected may vary.

For example, as illustrated in FIG. 9B, in the case in which the positive pressure is generated while the microneedle N penetrates by the final penetration depth of 4 mm and subsequently moves backward by 1 mm, the effective ingredients may be easily injected to a site from which the microneedle N is just withdrawn. In particular, the effective ingredients may be intensively injected around the depth of 3 mm.

In addition, the positive pressure may be maintained for a predetermined period of time even after the microneedle N has completely withdrawn from the skin. With this configuration, even after the microneedle N has completely withdrawn from the skin, the effective ingredients may be more easily injected to the site in the epidermal layer from which the microneedle N is withdrawn.

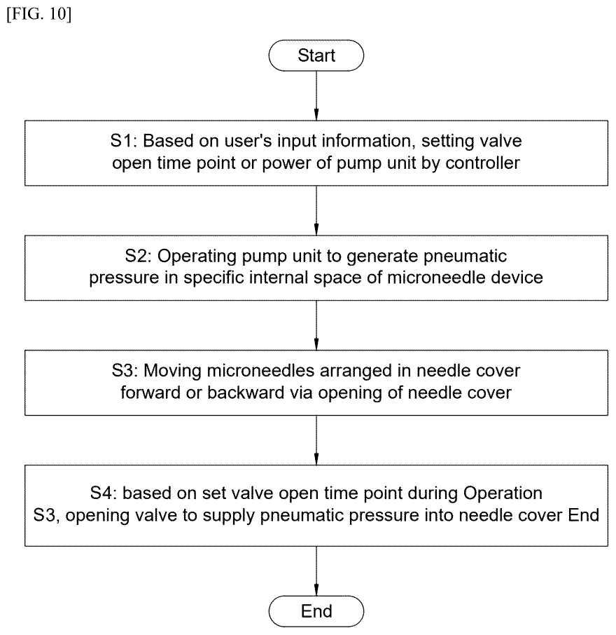

FIG. 10 is a flowchart exemplarily explaining a method of injecting effective ingredients into the skin using the microneedle device according to an example embodiment of the present disclosure.

As illustrated in FIG. 10, based on information input by the user (which is input using the display device and transmitted to the controller), the controller 400 may set a time point at which the valve is open or the power of the pump unit (Operation S1).

Subsequently, when the user presses the operation button of the operation unit 500, the pump unit 300 is operated to generate a pneumatic pressure in a specific internal space of the microneedle device (Operation S2). The microneedles N arranged in the needle cover 650 move forward or backward via the opening of the needle cover 650 (Operation S3).

When a time point at which the valve is set to be open reaches during Operation S3, the valve 430 is open to supply the pneumatic pressure generated by the pump unit 300 into the needle cover 650 (Operation S4).

### 4. Operation Mode of Microneedle Device According to Example Embodiment of Present Disclosure

### A. Manual Mode and Automatic Mode

As illustrated in FIG. 8, the microneedle device 10 according to an example embodiment of the present disclosure starts to operate when the user presses the operation button of the operation unit 500. That is, when the user presses the operation button, operation start information input by the user is transferred to the controller 400. The controller 400 controls the microneedles N to move forward and backward at an initial home position and return to the home position again, which is referred to one round of operation (needling operation). Further, the controller 400 controls the pump unit 300 and the valve 320 to be turned on and off during the needling operation according to a preset condition.

According to an example embodiment of the present disclosure, the microneedle device 10 may be set to perform the above-described operations each time the user presses the operation button of the operation unit 500 (in a manual mode). Alternatively, the microneedle device 10 may be set to perform the above-described operations automatically every set time interval when the user presses the operation button of the operation unit 500 (in an automatic mode).

As an example, the time interval in the automatic mode may be set in a range of 100 to 1,000 milliseconds (msec). For example, the time interval may be set to any one of 100, 200, 300, 400, 500, 600, 700, 800, 900, and 1,000 msec.

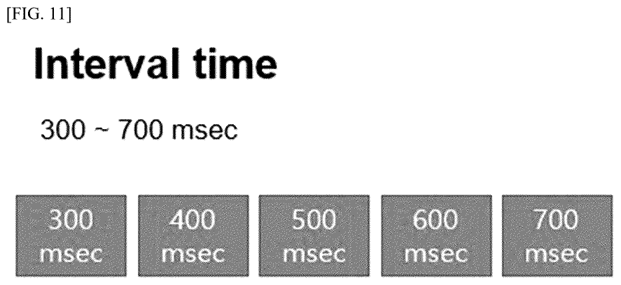

The display device 20 of the microneedle device 10 may provide the user interface 25 for receiving such setting information from the user. The setting information input from the user (that is, information about whether to operate in the manual mode or operate in the automatic mode, and time interval information in the case of the automatic mode) may be transmitted to the controller 400 of the microneedle device 10 via the cable 30. FIG. 11 exemplarily illustrates an example of the user interface for receiving the time interval information in a case where the time interval is set in a range of 300 to 700 msec.

### B. Stacking Mode

According to an example embodiment of the present disclosure, the penetration depths of the microneedles N in the automatic mode may be set to be different from each other according to the needling operation. For example, the penetration depths of the microneedles N may be set for each needling operation of a plurality of predetermined consecutive needling operations, which is referred to as a stacking mode. The stacking mode may be performed repeatedly.

According to an example embodiment of the present disclosure, in the stacking mode, the needling operation may be repeatedly performed twice or more times at the same skin position or at different time intervals.

According to an example embodiment of the present disclosure, the number of needling operations in the stacking mode may be variously set. For example, the number of needling operations may be selected in a range of two to ten times.

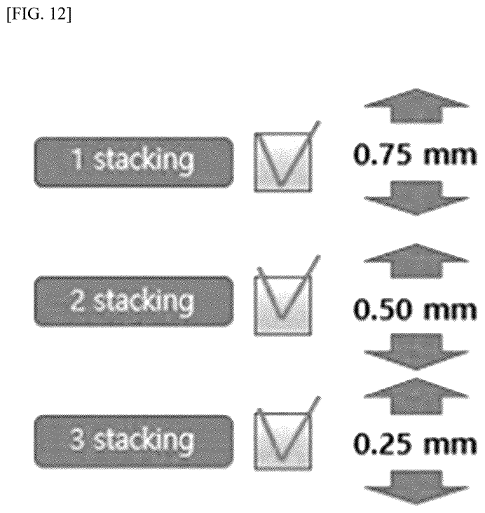

FIG. 12 exemplarily illustrates an example of the user interface for setting the penetration depth for each needling operation when the number of needling operations in the stacking mode is three. For the sake of convenience in description, in this specification, each needling operation in one stacking mode will be referred to as “stacking.” That is, in one stacking mode, a first round of needling operation will be referred to as “first stacking,” and a second round of needling operation will be referred to as “two stacking.”

As illustrated in FIG. 12, the penetration depth may be set for each stacking (needling operation). The penetration depth for each stacking may be set to be identical to that in a previous stacking, and may be set to be different from that in the previous stacking.

Referring to FIG. 12, the first stacking is set to perform the needling operation by the penetration depth of 0.75 mm. In this case, the user may increase or decrease the set penetration depth by clicking an upward arrow or a downward arrow. That is, at the time of the first stacking, the microneedles N move forward at the home position by the penetration depth of 0.75 mm and subsequently return to the home position. In the second stacking, the needling operation is set to be performed by the penetration depth of 0.50 mm. When the time interval is set to 100 msec, the second stacking is performed after 100 msec from when the first stacking is completed. That is, the microneedles N, which has been returned to the home position, move forward by the penetration depth of 0.50 mm and subsequently move backward to return to the home position. Similarly, a third stacking is performed after 100 msec from when the second stacking is completed. That is, the microneedles N move forward by the penetration depth of 0.25 mm and subsequently move backward to return to the home position. The stacking mode as described above may be repeatedly performed. For example, the first stacking may be performed again after 100 msec from when the third stacking is completed.

When the user presses the operation button of the operation unit 500 once in the stacking mode, the microneedles N may penetrate into the same skin position several times, which makes it possible to treat several layers in the skin (for example, a papillary layer, a plexiform layer and the like) at once by a mechanism of micro-needling and the injection of the effective ingredients. That is, the entire skin layer may be treated.

For example, in a case of a general mode other than the stacking mode, 100 rounds of needling operations are assumed to be performed at each of two different penetration depths (first and second penetration depths) in the entire facial skin. In this case, the penetration depth of the microneedle device is first set to the first penetration depth and the microneedle device needs to perform 100 rounds of needling operations while moving on the entire facial skin, and subsequently, the penetration depth of the microneedle device is set to the second penetration depth and the microneedle device needs to perform 100 rounds of needling operations while moving again along the same path on the entire facial skin. In contrast, in the case of the stacking mode, the first stacking may be set to be performed by the first penetration depth and the second stacking may be set to be performed by the second penetration depth. Thereafter, the microneedle device may perform 200 rounds of needling operations (that is, the stacking mode may be repeated 100 times) while moving once on the entire facial skin. With this configuration, it is possible to treat various layers of the skin once without moving the microneedle device several times along the same path on the entire facial skin.

Although the present disclosure has been described above in terms of specific items such as detailed constituent elements as well as the limited example embodiments and the drawings, they are merely provided to help more general understanding of the present disclosure, and the present disclosure is not limited to the above example embodiments. Various modifications and changes could have been realized by those skilled in the art to which the present disclosure pertains from the above description.

Therefore, the spirit of the present disclosure need not to be limited to the above-described example embodiments, and in addition to the appended claims to be described below, and all ranges equivalent to or changed from these claims need to be said to belong to the scope and spirit of the present disclosure.

### EXPLANATION OF REFERENCE NUMERALS

10: Microneedle device
20: Display device
25: User interface
30: Cable
100: Housing
200: Driver
210: Movable rod
300: Pump unit
310: Pneumatic hose
320: Valve
400: Controller
500: Operation unit
600: Needle assembly
610: Needle mounting plate
620: Fixture
630: Plunger
640: Connection tube
700: Needle cover
710: Space portion
720: Spring
800: Close-contact member
810: Through-hole
820: Axial extension portion
830: Recess
N: Microneedle

1. A microneedle device, comprising:
a housing;
a needle assembly including a plurality of microneedles;
a driver configured to generate a driving force for operating the needle assembly forward and backward;
a controller configured to control an operation of the microneedle device;
a needle cover configured to entirely or partially enclose the needle assembly; and
a close-contact member provided in a front portion of the needle cover and configured to bring into contact with a skin of a user when using the microneedle device,
wherein the close-contact member is formed of an elastically deformable material, and a front portion of the close-contact member is formed in a cup-like structure with a recess, and
wherein perforated portions are provided in a central portion of the close-contact member such that the plurality of microneedles move into and out of the microneedle device via the perforated portions.

2. The microneedle device of claim 1, wherein the close-contact member is configured to cover the front portion of the needle cover,
wherein the perforated portions of the close-contact member are formed as a plurality of through-holes provided in a central portion of the close-contact member configured to cover the front portion of the needle cover,
wherein the close-contact member includes an axial extension portion that extends in an axial direction, and
wherein the plurality of through-holes is provided in the axial extension portion.

3. The microneedle device of claim 2, wherein the axial extension portion is formed to extend backward from the close-contact member in the axial direction.

4. The microneedle device of claim 1, wherein the close-contact member is configured to be deformed when the microneedle device is pressed toward the skin of the user such that a bottom surface of the recess is brought into close contact with the skin of the user.

5. The microneedle device of claim 1, wherein each of the plurality of microneedles is formed in a tubular structure having a perforated central portion.

6. The microneedle device of claim 2, wherein the plurality of through-holes is provided to correspond in number to the plurality of microneedles provided in the needle assembly.

7. The microneedle device of claim 2, wherein each of the plurality of through-holes has a diameter of 0.25 mm to 3.0 mm.

8. The microneedle device of claim 1, further comprising:
a pump unit configured to generate compressed air and supply the compressed air to an interior of the needle assembly; and
a valve configured to open and close a pneumatic hose provided between the pump unit and the needle assembly.

9. The microneedle device of claim 8, wherein an opening/closing operation of the valve is controlled by the controller, and
wherein when the valve is brought into an open state, the compressed air is supplied from the pump unit to the interior of the needle assembly such that a positive pressure is formed in an inner space portion of the needle cover.

10. The microneedle device of claim 9, wherein the controller is configured to control the opening/closing operation of the valve based on information input from the user.

11. The microneedle device of claim 9, wherein the controller is configured to control power of the pump unit based on the information input from the user.

12. The microneedle device of claim 9, wherein, before the positive pressure is formed in the inner space portion of the needle cover, a negative pressure is formed in the inner space portion of the needle cover.

13. The microneedle device of claim 8, wherein the controller is configured to control at least one needling operation in which the plurality of microneedles is penetrated into the skin and subsequently return to original positions thereof; and
wherein the controller is configured to control the microneedle device in a stacking mode in which a penetration depth of each of the plurality of microneedles is set for each of the at least one needling operation.

14. The microneedle device of claim 13, wherein the stacking mode is set such that the at least one needling operation is repeated twice or more times at a same skin position or at different time intervals, and
wherein, in the stacking mode, the at least one needling operation is performed at a same penetration depth or different penetration depths.

15. A microneedle system, comprising:
the microneedle device of claim 8; and
a display device in communication with the controller of the microneedle device and configured to provide a user interface for receiving setting information about the operation of the microneedle device from a user and configured to transmit the setting information input from the user to the controller of the microneedle device.
